(19) United States
(12) Patent Application Publication
(10) Pub. No.: US 2024/0138984 A1
(43) Pub. Date: May 2, 2024

(54) DEVICE, SYSTEM, AND METHOD FOR INTRAVASCULARLY DELIVERING AN INTRAVASCULAR DEVICE TO A TARGETED CARDIAC VALVE

(71) Applicant: Cephea Valve Technologies, Inc., Abbott Park, IL (US)

(72) Inventor: Murrad Kazalbash, Santa Clara, CA (US)

(73) Assignee: Cephea Valve Technologies, Inc., Abbott Park, IL (US)

(21) Appl. No.: 18/492,285

(22) Filed: Oct. 23, 2023

Related U.S. Application Data
(60) Provisional application No. 63/381,057, filed on Oct. 26, 2022.

(51) Int. Cl. A61F 2/24

(52) U.S. Cl. CPC A61F 2/2436

(57) ABSTRACT

The present disclosure describes devices, systems, and methods for intravascularly delivering an intravascular device to a targeted cardiac valve.

### CROSS-REFERENCE TO RELATED APPLICATIONS

The present application claims priority to U.S. Provisional Ser. No. 63/381,057, filed Oct. 26, 2022, the disclosure of which is hereby incorporated by reference in its entirety as if fully set forth herein.

### BACKGROUND OF THE DISCLOSURE

Intravascular medical procedures allow the performance of therapeutic treatments in a variety of locations within a patient's body while requiring only relatively small access incisions. An intravascular procedure may, for example, eliminate the need for open-heart surgery, reducing risks, costs, and time associated with an open-heart procedure. The intravascular procedure also enables faster recovery times with lower associated costs and risks of complications. An example of an intravascular procedure that significantly reduces procedure and recovery time and cost over conventional open surgery is a heart valve replacement or repair procedure in which an artificial valve or valve repair device is guided to the heart through the patient's vasculature. For example, a catheter is inserted into the patient's vasculature and directed to the inferior vena cava. The catheter is then urged through the inferior vena cava toward the heart by applying force longitudinally to the catheter. Upon entering the heart from the inferior vena cava, the catheter enters the right atrium. The catheter may be guided across the atrial septum (e.g., via a guidewire that has already been passed through the atrial septum) into the left atrium. The distal end of the catheter may be deflected by one or more deflecting mechanisms in order to align the distal end of the catheter, as well as a medical device positioned therein, with the mitral valve. Catheter deflection can be achieved by tension cables, or other mechanisms positioned inside the catheter. Precise control of the distal end of the catheter allows for more reliable and faster positioning of a medical device and/or implant and other improvements in the procedures.

An intravascularly delivered device should be placed precisely to ensure a correct positioning of the medical device, which is important for its functionality, as the device may be difficult or impossible to reposition after the device is fully deployed from the delivery system. Additionally, the ability to recapture a partially deployed device is desirable in the event that the medical device is sub-optimally positioned and/or deployed.

### BRIEF SUMMARY OF THE DISCLOSURE

The present disclosure describes devices, systems, and methods for intravascularly delivering an intravascular (“IV”) device to a targeted cardiac valve.

A delivery system may include: a handle; a delivery member operably coupled to the handle and extending distally from the handle, the delivery member including an outer sheath, the outer sheath having a plurality of microfilaments braided together to form a braided coil; and a distal tip at a distal end of the delivery member, the distal tip being configured to receive a medical device therein.

In one example, the plurality of microfilaments may include a first microfilament, a second microfilament, and a third microfilament.

In one example, the first microfilament may have a first elasticity, the second microfilament may have a second elasticity less than the first elasticity, and the third microfilament may have a third elasticity less than both the first and second elasticities.

In one example, the plurality of microfilaments may be made of the same material.

In one example, the material may be nitinol.

In one example, the outer sheath may include a braided structure in addition to the braided coil, and a flexible cover that surrounds one or both of the braided structure and the braided coil.

In one example, the braided structure may surround the braided coil.

In one example, the outer sheath may include a second coil, and a flexible cover that surrounds one or both of the braided coil and the second coil.

In one example, when the outer sheath is in a bent condition, the plurality of microfilaments may be configured to slide over each to reduce recoil as the outer sheath translates relative to an interior catheter of the delivery member during medical device deployment.

In one example, each of the plurality of microfilaments may be circular in cross-section.

In one example, the delivery system may include the medical device, and the medical device may be a prosthetic mitral valve.

A segmented nosecone may include: a plurality of segments that, in an assembled condition, form the shape of a cone or a truncated cone, each of the plurality of segments including a positive alignment feature and a negative alignment feature configured to align the plurality of segments relative to each other; a plurality of lips, each of the plurality of lips hingedly coupled with a respective one of the plurality of segments via a respective hinge.

In one example, the plurality of segments, in the assembled condition, may form a tip defining a hole.

In one example, the plurality of segments, in the assembled condition, may define a lumen extending from a proximal end of the segmented nosecone to a distal end of the segmented nose cone.

In one example, a first portion of the lumen may define a continuously decreasing diameter from a proximal end to a distal end of the first portion and a second portion of the lumen defines substantially constant diameter.

In one example, the second portion of the lumen may define a substantially constant diameter approximately equal to an outer diameter of a guidewire.

In one example, each of the plurality of segments may include a locking ring.

In one example, the locking rings, in the assembled condition, may align longitudinally to define a locking ring lumen configured to allow a guidewire to pass therethrough.

A delivery system may include: a handle; a delivery member operably coupled to the handle and extending distally from the handle; a valve cover at a distal tip of the delivery member; and a segmented nose cone, the segmented nose cone being coupled to the valve cover by the plurality of lips.

A delivery system may include: a handle including a first gearset, a second gearset, and a control configured to bend a steering catheter via the first and second gearsets; and a delivery feedback assembly operably coupled to the control, the delivery feedback assembly configured to provide at least one of audible or tactile feedback while operating the control to bend the steering catheter.

In one example, the first gearset may include a hardstop mechanism configured to contact the second gearset to prevent further operation of the control in at least one direction.

In one example, the delivery feedback assembly may further include a plurality of feedback features configured to selectively engage with a projection of the hardstop mechanism, the selective engagement being configured to provide the at least one of audible or tactile feedback.

A bubble detection system may include: a cylinder configured to receive a catheter shaft; and an ultrasonic bubble sensor configured to receive the cylinder and detect a presence of air bubbles in the catheter shaft.

In one example, the system may include the catheter shaft, and the catheter shaft is a metal catheter shaft.

In one example, the cylinder may include: an inner wall and an outer wall, an interior portion of the inner wall defining a lumen configured to receive the catheter shaft, the inner wall and the outer wall together defining a fluid-tight chamber configured to hold an ultrasonic signal-propagating medium.

In one example, when the catheter shaft is received within the cylinder, the catheter shaft may not contact the ultrasonic signal-propagating medium.

In one example, as the catheter shaft moves relative to the lumen, that the ultrasonic bubble sensor may be configured to detect the presence of air bubbles along at least a portion of a length of the catheter shaft.

In one example, the movement of the catheter relative to the lumen may comprise at least one of movement of the catheter shaft or movement of a base to which the ultrasonic bubble sensor is mounted.

In one example, the cylinder may include a first hemostasis valve at a first end of the cylinder.

In one example, the cylinder may include a second hemostasis valve at a second end of the cylinder.

In one example, the cylinder may define a closed end at a second end of the cylinder.

### BRIEF DESCRIPTION OF THE DRAWINGS

FIG. 1 illustrates a delivery system configured for delivering, positioning, and deploying an IV device, the delivery system including a handle assembly coupled to a delivery member;

FIG. 2A illustrates a transverse cross-section of the delivery member showing various delivery member components that may be utilized, including a steering catheter and a delivery catheter disposed within and translatable within the steering catheter;

FIG. 2B illustrates a longitudinal cross-section of the delivery member;

FIG. 3 illustrates an exemplary approach for delivering an IV device to the mitral annulus;

FIG. 4 illustrates a distal end portion of the outer sheath, showing various sections that may be formed in the outer sheath;

FIG. 5 is a longitudinal cross-section of the outer sheath of FIG. 4;

FIG. 6 is a partial cut-away view of an intermediate portion of the outer sheath;

FIG. 7 illustrates a braided cable for use with a delivery member;

FIGS. 8A-8F illustrate views of a segmented nosecone for use with a delivery member;

FIGS. 9A-9F illustrate a delivery feedback assembly;

FIGS. 10A-B illustrate a bubble detection assembly;

FIG. 11 illustrates a bubble detection assembly according to another aspect of the disclosure; and

FIG. 12 illustrates a bubble detection assembly according to another aspect of the disclosure.

### DETAILED DESCRIPTION

As used herein, the term “proximal” when used in connection with a delivery system refers to the end of the delivery system closer to the user of the delivery system when being used in an intended manner, while the term “distal” refers to the end of the delivery system farther away from the user.

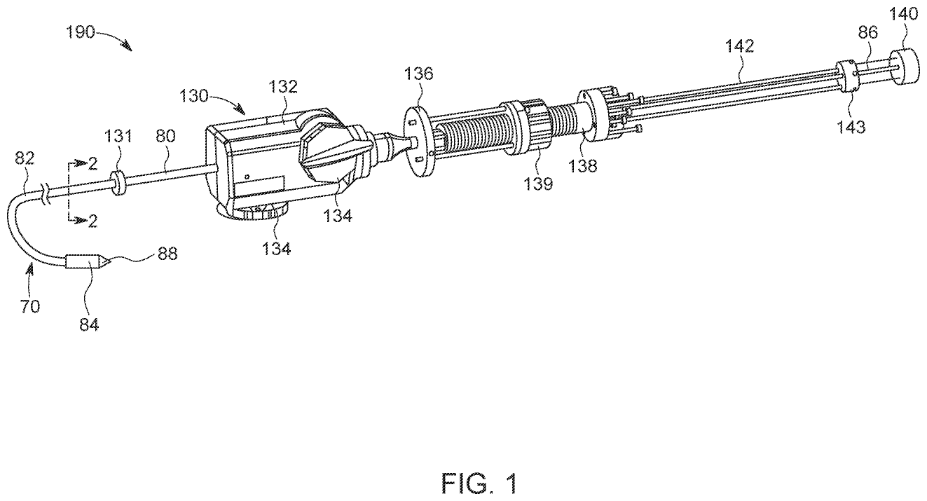

FIG. 1 illustrates an exemplary embodiment of a delivery system 190. As shown, the delivery system 190 includes a handle assembly 130 and a delivery member 70. The delivery member 70 is operably coupled to the handle assembly 130 and extends distally from the handle assembly 130. The delivery member 70 includes a plurality of catheter and/or hypotube members which provide different functionality during operation of the delivery system 190 to enable effective delivery and deployment of an IV device.

The proximal end of an outer sheath 82 is coupled to an end ring 131, and the outer sheath 82 extends to a distal tip 88. A steering catheter handle 132 is disposed proximal of the end ring 131. The proximal end of a steering catheter 80 is coupled to the steering catheter handle 132, and the steering catheter 80 extends distally from the steering catheter handle 132 into the outer sheath 82. The steering catheter handle 132 includes one or more controls 134 which are operably coupled to the steering catheter so that manipulation of the controls 134 adjusts the curvature of the steering catheter 80. For example, during delivery and deployment of a prosthetic mitral valve to a native mitral valve using a transseptal delivery route, the distal end of the delivery member 70 typically needs to be steered in at least two directions after clearing the atrial septum in order for the delivery member 70 (and the prosthetic mitral valve housed therein) to properly align with the native mitral valve.

The outer sheath 82 extends to a distal end where it is coupled to a distal piece 84 (which may also be referred to herein as a “valve cover 84”). The distal piece 84 functions to house an IV device in a compressed, pre-deployed state during intravascular delivery of the device to the targeted cardiac site.

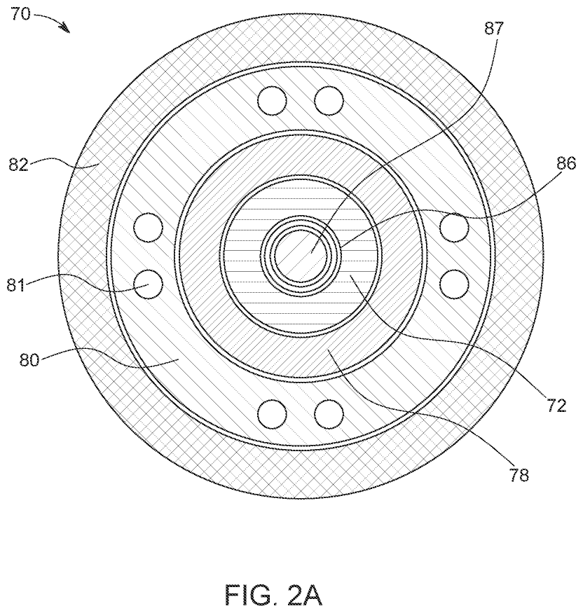

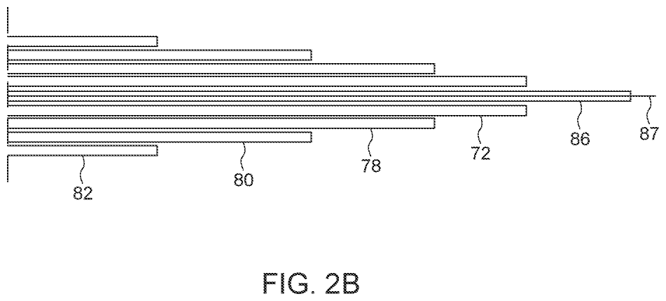

Because the steering catheter 80 is nested within the outer sheath 82, curving of the steering catheter 80 causes corresponding curving/steering in the outer sheath 82. The steering catheter 80 and outer sheath 82 may be referred to singly or collectively herein as the “outer member.” The illustrated embodiment of the delivery member 70 includes additional components which are not visible in the view of FIG. 1 but may be seen in the cross-sectional view of FIG. 2.

FIG. 2A illustrates a transverse cross-section of the delivery member 70 taken along the cross-section line 2-2 of FIG. 1. FIG. 2B illustrates a longitudinal cross-section of the delivery member 70. As shown, the steering catheter 80 is disposed radially within the outer sheath 82. A delivery catheter 78 (or alternatively referred to herein as an extension catheter) is disposed radially within the steering catheter 80. An inner catheter 72 (also referred to herein as suture catheter 72) may be disposed radially within the delivery catheter 78, and a guidewire tube 86 may be disposed radially within the inner catheter 72. The guidewire tube 86 is configured for receiving a guidewire 87. Although the particular nested configuration shown in FIGS. 2A-B represents one preferred embodiment, alternative embodiments may include a different concentric arrangement of constituent parts. For example, some embodiments may combine the steering catheter 80 and outer sheath 82 and/or configure the outermost member with steering functionality, some embodiments may include more than one catheter with steering functionality, etc.

The steering catheter 80 is configured to be selectively curved to facilitated intravascular navigation. In some embodiments, the steering catheter 80 provides steerability via a plurality of lumens 81 extending through the length of the wall of the steering catheter 80. The lumens 81 may be configured for receiving tension cables or pull wires which extend between the controls 134 and a steering ring at or near the distal end of the steering catheter 80. One or more tension cables may additionally or alternatively be coupled to intermediate sections of the steering catheter 80. Manipulation of the controls 134 therefore adjusts tension in the tension cables to increase or decrease curvature of the steering catheter 80 at various positions. In the particular example shown in FIG. 2A, each tension cable is in a looping configuration in which two ends of the steering cable are coupled to controls 134, with a middle or intermediate section of the steering cable looping around the steering ring at or near the distal end of the steering catheter 80. Thus, referring still to FIG. 2A, two pairs of diametrically opposed lumens 81 provide for steering in a first plane, while the other two pairs of diametrically opposed lumens 81 provide for steering in a second plane substantially orthogonal to the first plane. Although the controls 134 are shown here as knobs, alternative embodiments may additionally or alternatively include one or more buttons, sliders, ratcheting mechanisms, or other suitable controls capable of adjusting tension to provide steering. Illustrative structures that can be used as part of the steering catheter handle 132 and or steering catheter 80 are described in U.S. Pat. No. 7,736,388, which is incorporated herein by this reference.

Referring again to FIG. 1, a delivery catheter holder 136 is disposed proximal of the steering catheter handle 132. Although not visible in the view of FIG. 1, the proximal end of the delivery catheter 78 is coupled to the delivery catheter holder 136. The delivery catheter 78 extends distally away from the delivery catheter holder 136 and into the steering catheter 80. An inner catheter holder 138 (also referred to herein as suture catheter holder 138) is disposed proximal of the delivery catheter holder 136. The inner catheter 72 may be coupled to the inner catheter holder 138 so that translation of the inner catheter holder 138 corresponds to translation of the inner catheter 72. For example, the inner catheter 72 may be selectively locked relative to the inner catheter holder 138 through a set screw, clamp, or other selective holding mechanism. The inner catheter 72 extends distally away from the inner catheter holder 138 and into the delivery catheter 78.

An inner catheter control 139 is operatively coupled to the inner catheter holder 138. Manipulation of the inner catheter control 139 adjusts the relative positioning of the delivery catheter holder 136 and inner catheter holder 138, and thus the relative positioning of the delivery catheter 78 and the inner catheter 72. In the illustrated embodiment, the inner catheter control 139 operates through threaded engagement with the inner catheter holder 138, such that rotation of the inner catheter control 139 translates the inner catheter holder 138 relative to the control 139 and therefore relative to the delivery catheter holder 136. Alternative embodiments may additionally or alternatively include one or more of a slider and rail assembly, a ratcheting mechanism, or other suitable means of linear adjustment.

The inner catheter 72 may extend proximally to and be attached to an inner catheter cap 143. A user may decouple the inner catheter 72 from the inner catheter holder 138 to allow movement of the inner catheter 72 by sliding/translating the inner catheter cap 143 along alignment rods 142. The guidewire tube 86 extends distally through the alignment cap 143 and into the inner catheter 72. The guidewire tube 86 extends to the distal end of the delivery member 70 where it is attached to a distal tip 88. The distal tip 88 is preferably formed from a flexible polymer material and provides an angled, atraumatic shape which assists in passing the delivery member 70 through the vasculature without tearing or otherwise damaging the patient's tissue as the leading end of the delivery member 70 comes into contact with tissue. The distal tip 88 may also facilitate the leading end of the delivery member 70 passing through the inter-atrial septum to the mitral annulus, which is required in a typical transfemoral approach to the mitral annulus.

In the illustrated embodiment, the guidewire tube 86 is coupled to a guidewire tube holder 140. By moving the guidewire tube handle, the guidewire tube 86 may be selectively translatable relative to the inner catheter cap 143 such that the guidewire tube 86 and distal tip 88 may be linearly translated relative to the inner catheter 72 and other components of the delivery member 70. The guidewire tube 86 may be selectively locked in a longitudinal position relative to the inner catheter holder 138 and/or inner catheter cap 143, such as through a set screw, clamp, or other selective fastener. For example, such a fastening structure may be associated with the inner catheter cap 143.

When unlocked, the guidewire tube 86 (and likewise the distal tip 88) may be moved relative to the inner catheter 72. The ability to retract the distal tip 88 relative to the inner catheter 72 reduces the risk that the distal tip 88 will become overextended during deployment, where it could become tangled in chordae tendineae and/or cause injury to cardiac tissue. Additionally, independent movement of the guidewire tube 86 (with the distal tip 88) also allows for closing the gap between the distal tip 88 and the valve cover 84 following deployment of the intravascular device. When the intravascular device has been released, the distal tip 88 is separated from the valve cover 84 by a distance, such as by about 40 mm. To avoid drawing air into the catheter, the gap between valve cover 84 and distal tip 88 is closed by drawing the distal tip 88 towards the valve cover 84, preferably in the left side of the heart, to avoid sucking air into the catheter when pulled back into the right side of the heart (where there is relatively low pressure).

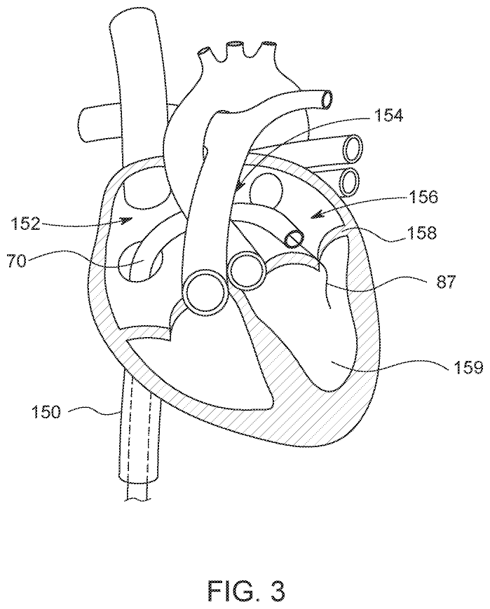

FIG. 3 illustrates a schematic representation of a patient's heart (shown in a cut-away view) and a delivery procedure to the mitral annulus that may be conducted using the illustrated delivery system 190. The delivery member 70 may be inserted into the patient's vasculature (e.g., through the femoral vein in a transfemoral approach) and directed to the inferior vena cava 150. The delivery member 70 is passed through the inferior vena cava 150 toward the heart. Upon entering the heart from the inferior vena cava 150, the delivery member 70 enters the right atrium 152. For procedures relating to the mitral valve 158, the delivery member 70 is further passed into the left atrium 156 by passing through a puncture in the inter-atrial septum 154. The puncture in the inter-atrial septum 154 may be created in a separate standard procedure, not described in more detail herein, prior to inserting the delivery system 190 into the patient. During this separate standard procedure, the guidewire 87 may be placed along the desired delivery pathway and through the atrial puncture, extending into the left atrium 156. If desired, the guidewire 87 may be further passed through the mitral valve 158 and into the left ventricle 159, as shown in FIG. 3, to provide a rail over which the delivery system 190 may ride.

In other implementations, such as for procedures associated with a tricuspid valve, the delivery member 70 may be passed through the inferior vena cava 150 and into the right atrium 152, where it may then be positioned and used to perform the procedure related to the tricuspid valve (i.e., the right atrioventricular valve). As described above, although many of the examples described herein relate to delivery to the mitral valve, one or more embodiments may be utilized in other cardiac procedures, including those involving the tricuspid valve.

Although a transfemoral approach for accessing a targeted cardiac valve is one preferred method, it will be understood that the embodiments described herein may also be utilized where alternative approaches are used. For example, embodiments described herein may be utilized in a transjugular approach, transapical approach, or other suitable approach to the targeted anatomy. For procedures related to the mitral valve or tricuspid valve, delivery of the artificial, replacement valve or other IV device is preferably carried out from an atrial aspect (i.e., with the distal end of the delivery member 70 positioned within the atrium superior to the targeted cardiac valve). The illustrated embodiments are shown from such an atrial aspect. However, it will be understood that the IV device embodiments described herein may also be delivered from a ventricular aspect.

In some embodiments, a guidewire 87 is utilized in conjunction with the delivery member 70. For example, the guidewire 87 (e.g., 0.014 in, 0.018 in, 0.035 in) may be received within the guidewire tube 86 of the delivery member 70 as the delivery member 70 is advanced over the guidewire 87 toward the targeted cardiac valve.

Additional details regarding delivery systems and devices that may be utilized in conjunction with the components and features described herein are described in United States Patent Application Publication Numbers 2018/0028177A1 and 2018/0092744A1, which are incorporated herein by this reference.

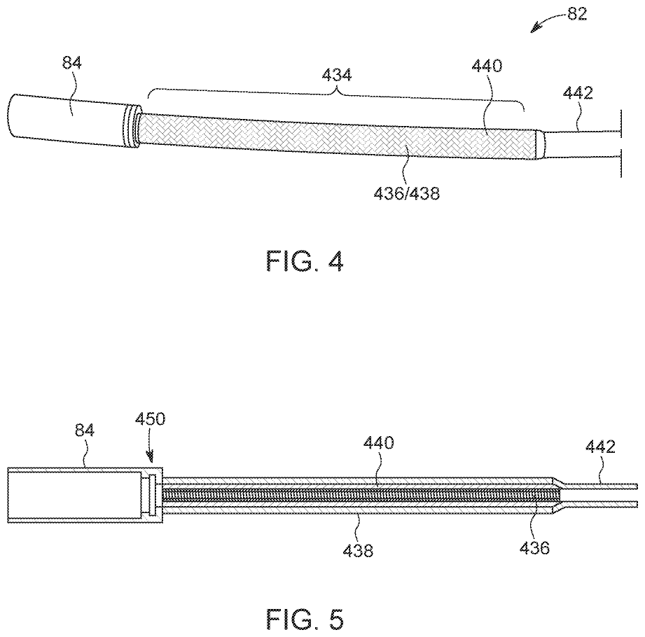

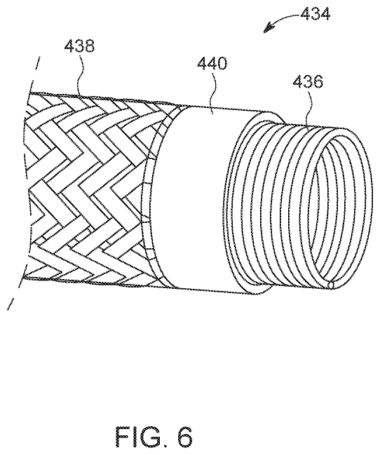

FIGS. 4 and 5 illustrate a portion of the distal end of the outer sheath 82 and distal piece 84 (also occasionally referred to herein as cover 84). Distal piece 84 can be formed as a cylindrical tube having an inner diameter and length sized to receive the IV device, in a collapsed/pre-deployed configuration, within the lumen of distal piece 84. For example, the IV device may be a collapsible and expandable prosthetic heart valve, which may include a self-expanding anchoring frame coupled to a self-expanding valve frame, with a plurality of prosthetic leaflets coupled to the valve frame to provide the valve functionality. Distal piece 84 can include a plurality of microfabricated cuts (e.g., laser cuts) and a pair of continuous longitudinal spines located on opposite sides so that distal piece 84 can bend and flex substantially in a single plane. The outer sheath 82 can also include a bending portion 434 that can be attached to and located proximal to distal piece 84. Bending portion 434 may have a sufficient length to surround and extend along that portion of the delivery system that is designed to bend and reorient, via the steerable catheter 80, to navigate through a patient's vasculature and/or heart to a target site for deploying the IV device. In the context of a transfemoral mitral valve procedure, the bending portion 434 may generally correspond to portions of the outer sheath 82 that extend through the atrial puncture and within the right atrium during deployment of the prosthetic mitral valve. In some embodiments, the bending portion 434 can include a cable tube or coil 436 surrounded by a braided structure 438 (sometimes collectively referred to as the “coil/braid portion 436/438”) as shown in FIG. 6.

Attached to the proximal end of bending portion 434 is a cut hypotube 442 that extends from bending portion 434 to the proximal end of the sheath 82. Hypotube 442 can include a plurality of slits and at least one longitudinally continuous spine that can preferably be continuous and uninterrupted along a longitudinal length of, and located at a fixed angular location on, hypotube 442.

In such embodiments, it can be desirable for the bending portion 434 of delivery catheter to remain liquid tight. To seal the bending portion 434, a flexible, fluid impermeable covering can be provided over the coil/braid portion 436/438, extending from the distal piece 84 to a location proximal the coil/braid portion 436/438. For example, the delivery sheath 82 can also include a thin-walled flexible cover 440 that extends from the distal piece 84 to the hypotube 442. Flexible cover 440 can be bonded at each end to the underlying structure, using one of a variety of different adhesives, thermal adhesives, UV bonded adhesive, or other techniques.

Referring again to FIG. 5, outer sheath 82 can also be coupled to distal piece 84 via a swivel connection, generally indicated at 450. To overcome the challenging forces that can develop during insertion of a relatively large delivery catheter into the vasculature of a patient, swivel connection 450 allows rotation of outer sheath 82 by a few degrees, back and forth (i.e., alternating between clockwise rotation and counter-clockwise rotation) while at the same time moving the delivery system 190 in a generally longitudinal direction. This rotational motion (during simultaneous longitudinal translation) helps to overcome some of the longitudinal forces that may resist insertion of outer sheath 82 through a patient's vasculature or frictional forces between the outer sheath 82 and the steering catheter 80.

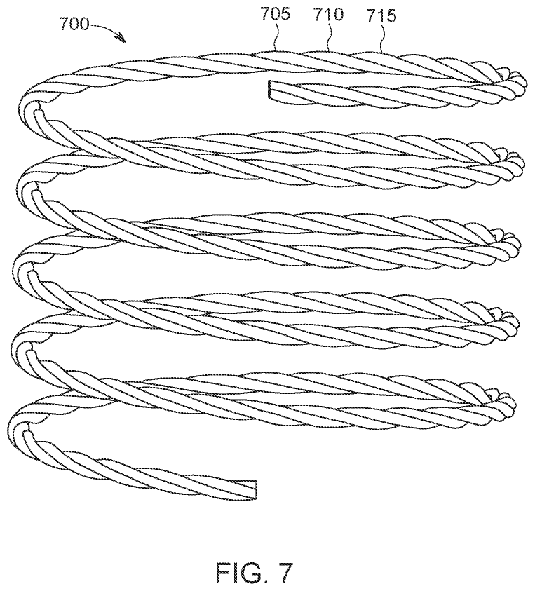

FIG. 7 illustrates a braided coil 700 for use with a delivery member. For example, the braided coil 700 can be used with delivery member 70.

In certain existing systems that incorporate a flat wire, such as cable tube or coil 436 depicted in FIG. 6, the flat profile of the wire may not provide adequate compressive strength or angular dampening as the surfaces are smooth and the recoil energy cannot dissipate elsewhere. This can result in abrupt recoil in areas where the outer catheter coil bunches up and interacts with the steering catheter during bending operations. For example, during deployment of a prosthetic mitral valve from the valve cover 84, outer sheath 82 is typically withdrawn proximally relative to other interior catheters, such as the suture catheter 72 which is coupled to the prosthetic mitral valve. During this proximal translation of the outer sheath 82 relative to the prosthetic mitral valve, the bending portion 434 of the outer sheath 82 typically has significant bending, including for example about 90 degrees bend between the atrial puncture and the center of the native mitral valve. As the outer sheath 82 withdraws, coil 436 may compress and/or frictionally engage the outer surface of the steering catheter 80. If the entire bending portion 434 were perfectly straight during such retraction, the retraction may not be significantly likely to move in anything other than a controlled manner. However, the significant bends that the outer sheath 82 follows as it is withdrawn during deployment of the prosthetic heart valve may tend to result in “jumping” or other abrupt and undesirable movement of the outer sheath 82. If the flat wire that forms the coil 436 does not provide smooth movement of the outer sheath 82 during withdrawal, and the outer sheath 82 “jumps” during retraction, the deployment of the prosthetic mitral valve may end up being somewhat uncontrolled, which may sacrifice the ability to properly deploy and position the prosthetic mitral valve.

As shown in FIG. 7, the braided coil 700 can include a plurality of individual microfilaments, e.g., 705-715. In one example, the braided coil 700 includes a first microfilament 705, a second microfilament 710, and a third microfilament 715. In other examples, the braided coil 700 can include at least two microfilaments. A thickness of each of the microfilaments and, ultimately, a thickness of the coil 700 can vary according to the application. In one example, the thickness of each of the microfilaments 705-715 can be identical, while in other examples the thicknesses can differ. Depending on a desired length of the braided cable coil, a thickness of the overall coil 700 can vary. For example, for a longer length (corresponding to a full-length shaft), the coil 700 can be thicker. On the other hand, for a shorter length (corresponding to a portion of a shaft length), the coil 700 can be thinner.

The microfilaments 705-715 can be braided, or weaved together, to form a single monolithic braided coil 700. The microfilaments 705-715 can be made from the same or different materials. In one example, the microfilaments 705-715 can each be made of nitinol. In other examples, each of the microfilaments 705-715 can be made from different materials or any subset of the microfilaments 705-715 can be made of the same material. In some examples, one, some, or all of the microfilaments 705-715 may have a circular shape, or generally circular shape, in transverse cross-section. This is, for example, in contrast to a flat wire that has a generally rectangular shape in transverse cross-section.

In one particular example, the microfilaments 705-715 can be formed of three different materials representing different elasticities. One microfilament can have a first elasticity, another microfilament can have a second elasticity less than the first elasticity, and still another microfilament can have a third elasticity less than both the first and second elasticities.

Referring to FIG. 6, the bending portion 434 can include a cable tube or coil 436 surrounded by a braided structure 438 and a flexible cover 440.

In one example, the cable tube or coil 436 can incorporate the braided coil 700 and the bending portion can include braided coil 700, braided structure 438 that surrounds the braided coil 700, and flexible cover 440 that surrounds one or both of the braided coil 700 and the braided structure.

In another example, the braided structure 438 can incorporate the braided coil 700 and the bending portion can include coil tube or coil 436, braided coil 700, and flexible cover 440 that surrounds one or both of the coil tube or coil 436 and braided coil 700.

In still another example, the bending portion can include a braided coil 700 and a flexible cover 440 that surrounds the braided coil 700 or is surrounded by the braided coil.

Advantageously, the braided coil 700 provides both flexibility and rigidity. Incorporation of the braided coil 700 as described above can prevent the abrupt recoil described above during bending operations by allowing the microfilaments to slide over each other such that each microfilament glides over and provides microscopic relief in tension, resulting in microdampening and reduction of the abrupt recoil. And while the braided coil 700 may still provide adequate column strength similar to a flat-wire, non-braided coil, the multi-filament braid structure increases surface area contact with other components of the delivery system (e.g., the outer surface of steering catheter 80), as well as increasing surface area contact with itself. In other words, the different microfilaments 705-715 contact each other and provide “grip” that results in reduced bunching of the coil 700, compared to a single flat-wire coil. By avoiding this bunching, there is little or no opportunity for the bunched area(s) of the coil to rapidly relax or decompress, which could result in the undesirable “jumping” or “skipping” of the outer sheath 82 as it is retracted during deployment. In this regard, the braided coil can function as a deflection recoil dampener and column strength provider. The intertwined filaments in a coil shape allow for controlled flexing and unflexing of a steerable catheter while simultaneously providing hoop strength. The nature of the wrapped wires provides a slower transition when relaxing a bend or sliding this member over another flexed shaft. The braided coil also advantageously serves as a strain relief, maintaining pitch more reliably than a simple coil.

In certain existing systems, assembly of the nosecone and/or any corresponding nosecone catheter assembly can be challenging during clinical device preparation, as such assembly requires dexterity of handling small parts and threading to a catheter underwater. As an example, for the delivery system 190 shown in FIG. 1, the distal tip 88 (which may be referred to as a nosecone) is only coupled to the remainder of the delivery system 190 during or after loading of the prosthetic heart valve or other IV device within the valve cover 84. In one example, the nosecone 88 includes a catheter shaft, which may be referred to as a nosecone catheter, that is coupled (e.g., threaded) onto a distal end of the guidewire tube 86 to couple the nosecone 88 to the delivery system 190. In that example, the nosecone catheter includes a lumen such that the guidewire 87 can pass from the guidewire tube 86 to the lumen of the nosecone catheter and vice versa. However, the requirement of assembling the nosecone 88 and/or the corresponding nosecone catheter to the guidewire tube 86 may be manually challenging and may need to be performed in a saline bath to try to avoid air entering the delivery system 190 during assembly. This may increase the time required for the procedure, as well as the complexity of the procedure, since the nosecone 88 is not pre-assembled to the remainder of the delivery system 190.

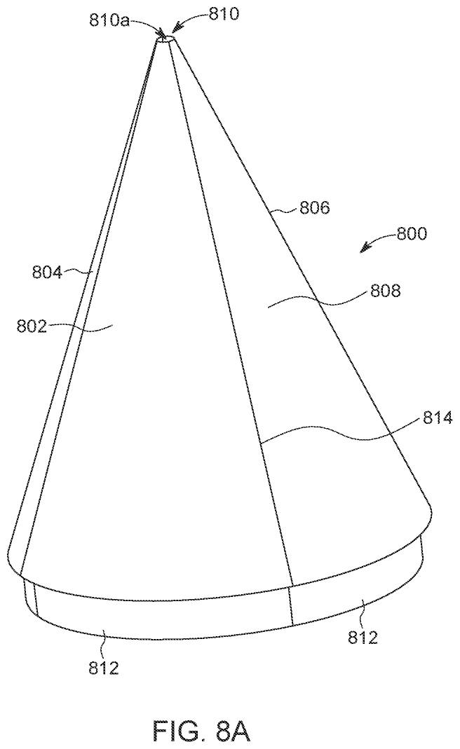

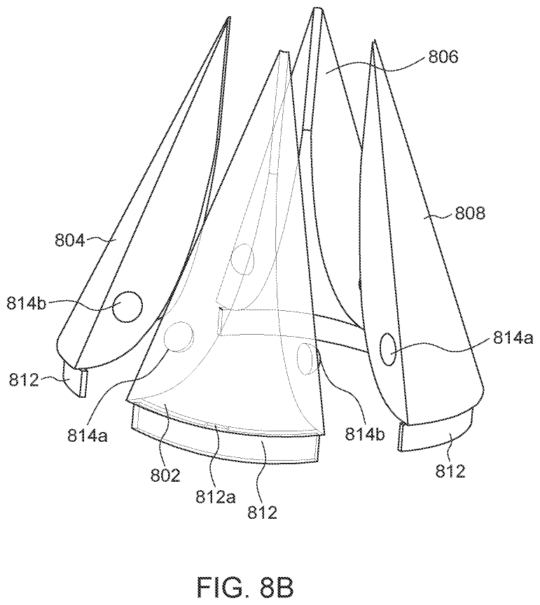

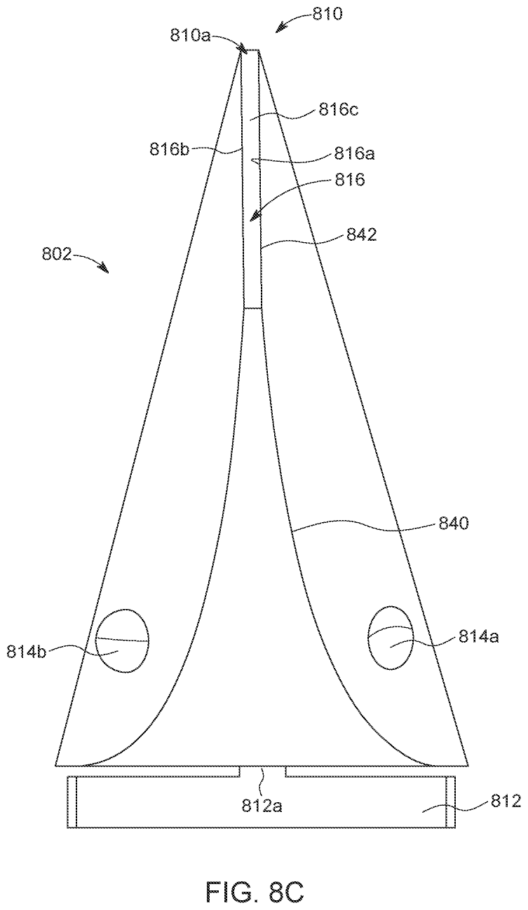

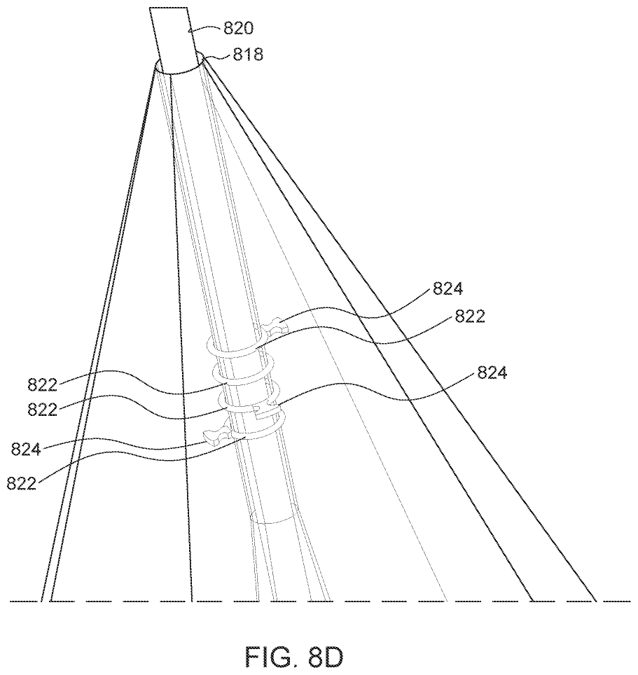

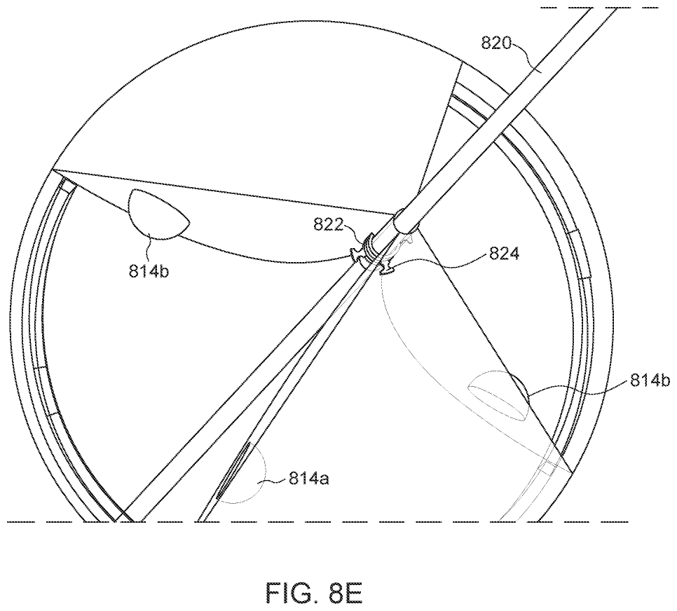

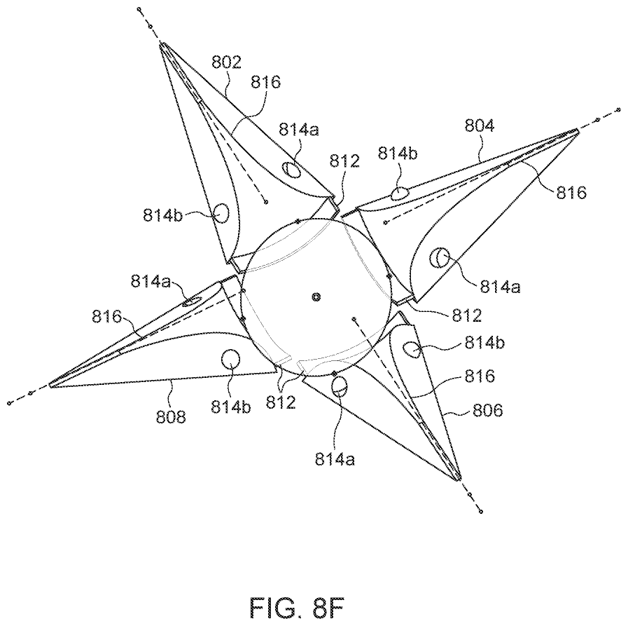

FIGS. 8A-8G illustrate views of a segmented nosecone 800 for use with a delivery member 70. Advantageously, the segmented nosecone 800 may reduce an overall French size of a delivery system by eliminating the above-described nosecone catheter. Further, the segmented nosecone 800 may use the guidewire 87 to hold segments of the nosecone 800 in an assembled state. Even further, the segmented nosecone 800 can accommodate a portion of the valve to be implanted, which thereby shortens the required length of the valve cover (e.g., valve cover 84 described above). It may generally be desirable for the valve cover 84 to be as short as feasible because the valve cover 84, particularly when the prosthetic heart valve (or other IV device) is positioned therein, is highly rigid and unable to bend. A relatively long valve cover 84 may create difficulties in maneuvering the valve cover into a desired (generally coaxial) orientation within the native mitral valve annulus once the valve cover 84 traverses the atrial septum, as the valve cover typically needs to turn about 90 degrees after clearing the atrial septum. This maneuvering becomes more difficult as the length of the valve cover 84 increases since there is only a limited amount of space within the left atrium for such maneuvering to occur. Even further, by eliminating the above-described nosecone catheter, as well as the requirement to couple the nosecone and/or nosecone catheter to the delivery system while preparing the delivery system for use, the procedural time and complexity (and cost) may be reduced.

FIG. 8A illustrates the segmented nosecone 800 with segments 802-808 in an assembled condition relative to one another, while FIG. 8B illustrates an exploded view of the segments 802-808 of the segmented nosecone 800 with segment 802 shown partially in phantom to better illustrate the remaining segments 804, 806, 808. The segmented nosecone 800 can be any type of material. However, the material forming the nosecone 800 is preferably one that, in combination with the shape of the nosecone, will not cause trauma to the blood vessels it passes through. Such materials may include soft, low durometer materials. The segmented nosecone 800 can be positioned at a distal tip of delivery member 70 (e.g., at the distal portion of the valve cover 84 in place of the previously described nosecone 88). In this regard, the segmented nosecone 800 can affix directly or indirectly to the valve cover 84, without the need for a separate nosecone catheter to couple the nosecone 800 to the delivery system 190. In one example, the lip 812 of the segmented nosecone 800, described in greater detail below, is bonded directly to the valve cover 84.

As shown in FIG. 8A, the segmented nosecone 800 can include a plurality of segments 802-808 which, when assembled, can generally form the shape of a cone or a truncated cone defining a plurality of seams 814. At a distal end of the segmented nosecone 800 is a tip 810 defining a hole 810a and at a proximal end of the nosecone 800 is a lip 812 for engaging with the valve cover 84. When in the assembled condition of FIG. 8A, the nosecone 800 may serve as, inter alia, a leading atraumatic tip of the delivery system 190.

As shown in FIG. 8B, each of the segments 802-808 can have one or more engagement and/or alignment features 814 a,b. In one example, each of the segments 802-808 includes at least one positive alignment feature 814b (e.g., a projection, such as a portion of a sphere) and at least one negative alignment feature 814a (e.g., a detent or recess, such as a recess complementary to the portion of the sphere of the positive alignment feature) such that, when the segments 802-808 are being assembled the positive alignment features 814b are received within or otherwise engage with the negative alignment features 814a to align the segments 802-808 relative to one another. In the example where the alignment features are a projection and recess, the projection securely fits within the recess in the assembled state.

Each of the segments 802-808 can be hingedly coupled to a lip 812 via a hinge 812a. Each of the segments 802-808 can be integrally formed with the respective lip 812 and hinge 812a, while in other examples, one or more of the segment, lip, and hinge can be individual elements. In the illustrated embodiment, each hinge 812a is in the form of a living hinge or flexure bearing. However, in other embodiments, each hinge 812a may be any type of hinge that allows the lip to pivot relative to the respective segment. Each of the lips 812 can be a portion of a cylinder (e.g., 90 degrees or a fourth of a cylinder) such that, when assembled, the lips 812 collectively define a cylinder defining an internal and external diameter. In the assembled state, the external diameter of the assembled lips 812, may be smaller than a maximum external diameter of the assembled segments 802-808. In other words, the assembled lips 812 may form an exterior shoulder with the assembled segments 802-808. The lip(s) 812 can attach directly or indirectly to a valve cover (e.g., valve cover 84). For example, the outside of the cylinder formed by lips 812 may fit snugly within the inner diameter of the valve cover 84 at the distal end of the valve cover 84. In this regard, when coupled to the valve cover, the segments 802-808 can each individually flex or rotate about the respective hinges 812a.

FIG. 8C illustrates a side view of one segment 802. While only one segment 802 is depicted, it is understood that each of the segments 802-808 can have a similar or an identical structure.

Each segment 802 can define a longitudinal channel 816 extending from a proximal end to a distal end of the segment 802 and between opposing portions 816a, b of an interior wall 816c such that, when the segments 802-808 are assembled, the respective channels 816 of each of the segments 802-808 align to define a single lumen or conduit 818 extending from the proximal end to the distal end of the nosecone 800 and configured to receive a guidewire 820.

When the segments 802-802 are in an assembled condition, similar to that shown in FIG. 8A, the longitudinal channel 816 can have a first larger diameter portion 840 for receiving a portion of the prosthetic heart valve and a second smaller diameter portion 842 for receiving the guidewire 87 therethrough. The first portion 840 can be proximally located relative to the second portion 842. The first portion 840 can have a continuous taper such that a diameter defined by opposing portions 816a, b of interior wall 816c is continuously decreasing from a proximal end to a distal end of the first portion 840. The second portion 842 can have a substantially constant diameter that is approximately equal to the outer diameter of the guidewire 87 that will be received through the second portion 842.

FIG. 8D is a partial view illustrating the nosecone 800 in an assembled state and partially in phantom, depicting a guidewire 820 (which may be the same as guidewire 87) and locking rings 822, and FIG. 8E is a top perspective view illustrating the nosecone 800 in an assembled state and partially in phantom, depicting a guide wire 820 and locking rings 822.

As shown in FIG. 8D, each of the segments 802-808 can include at least one locking ring 822 that is attached to interior wall 816c via an attachment feature 824. The attachment feature 824 can permanently affix the locking ring 822 with respect to interior wall 816c of the segment such that the guidewire 820 can pass through each of the locking rings 822. In some embodiments, the locking rings 822 are formed integrally with each corresponding segment 802-808, for example via injection molding. If the locking rings 822 are formed integrally with the segments 802-808, the attachment feature 824 may not be a separate physical structure. In other examples, the locking rings 822 are formed separately and then attached, via attachment feature 824, to the corresponding segment 802-808.

As shown in FIG. 8D, each segment 802-808 can have at least one locking ring 822 such that, in an assembled state, the at least four locking rings 822 align longitudinally, defining a locking ring lumen, to allow the guidewire 820 to pass through all of the locking rings 822 simultaneously. When the segments 802-808 are in the assembled condition, the locking ring lumen may be slightly smaller in interior diameter than, and concentric or coaxial with, the interior diameter of the second portion 842 of the longitudinal channel 816.

FIG. 8F is a top view illustrating the segments 802-808 during expression of the valve. As used herein, the phrase “expression of the valve” generally refers to the expansion (e.g., self-expansion) of the prosthetic heart valve as the valve cover 84 is withdrawn relative to the collapsed prosthetic heart valve. While the valve cover 84 covers the prosthetic heart valve, the valve cover 84 constrains the prosthetic heart valve (if it is a self-expanding prosthetic heart valve). As the valve cover 84 is withdrawn proximally relative to the prosthetic heart valve, and the constraint is removed, the prosthetic heart valve begins to self-expand (or “express”) while it attempts to return to its set-shape.

In a typical operation of a delivery system 190 that incorporates nosecone 800 in place of nosecone 88, access to the cardiac site (e.g., the left or right atrioventricular valve) is established first with a guidewire, such as guidewire 87 or 820. If not previously done, the prosthetic heart valve (or other IV device) is positioned, e.g., in a collapsed condition, entirely or partially within the valve cover 84. With guidewire access established, and the guidewire 820 extending from the target site to a location outside the patient's body, the segments 802-808 of the nosecone 800 may be brought together and passed over the proximal end of the guidewire 820 so that the guidewire 820 extends through each of the locking rings 822. The contact between the guidewire 820 and the locking rings 822 helps to ensure that the segments 802-808 remain in the assembled condition as long as the guidewire 820 is positioned within the locking rings 822. The delivery device is advanced over the guidewire 820 until reaching the desired target site of the heart. When the operator is ready to deploy the prosthetic heart valve from the valve cover 84. the guidewire 820 is pulled back proximally until the none of the locking rings 822 are occupied by the guidewire 820. When the guidewire 820 is pulled back, it retracts through the locking rings 822, releasing the constraint that otherwise restricts the segments 802-808 from “opening” or otherwise moving away from each other.

When the prosthetic heart valve is received within the valve cover 84, less than the entirety of the prosthetic heart valve may be positioned within the valve cover 84. Rather, a short distance of the prosthetic heart valve may extend distally beyond the end of the valve cover 84, with that short distance being received within the first portion 840 of the longitudinal channel 816 such that a leading portion of the prosthetic heart valve is in direct contact with the interior wall 816c. Because the prosthetic heart valve is self-expandable, it will always exert a radial outward force on any structures maintaining the prosthetic heart valve in a collapsed condition. Thus, during delivery, the prosthetic heart valve, and in particular the length of the prosthetic heart valve received within the first portion 840 of the longitudinal channel 816, exerts a radial outward force on each of the segments 802-808. However, as long as the guidewire 820 remains within the locking rings 822, the outward radial force on the segments 802-808 will not cause the segments to flex outwardly about hinges 812a. As noted above, when it is time to deploy the prosthetic heart valve, the guidewire 820 is retracted proximally until it fully clears all of the locking rings 822. No longer having the lateral holding force provided by the locking ring 822 engagement with the guidewire 820, each of the segments 802-808 can flower outward (as shown in FIG. 8F) by virtue of the outward forces from self-expansion of the prosthetic heart valve and/or the freedom of motion provided by hinge 812a. Since each respective lip 812 is fixed to the valve cover 84, the outward flowering can also be described as a pivoting motion of each respective segment 802-808 relative to its respective lip 812 via hinge 812a.

Once the segments 802-808 flower open, as shown in FIG. 8F, the valve cover 84 can be withdrawn proximally to expose a remaining portion of the prosthetic heart valve or other IV device within the valve cover 84. The segments 802-808 may remain in the flowered open state during withdrawal of the valve cover 84 and without interfering with the withdrawal of valve cover 84 and/or delivery of the prosthetic heart valve or other IV device within the valve cover 84.

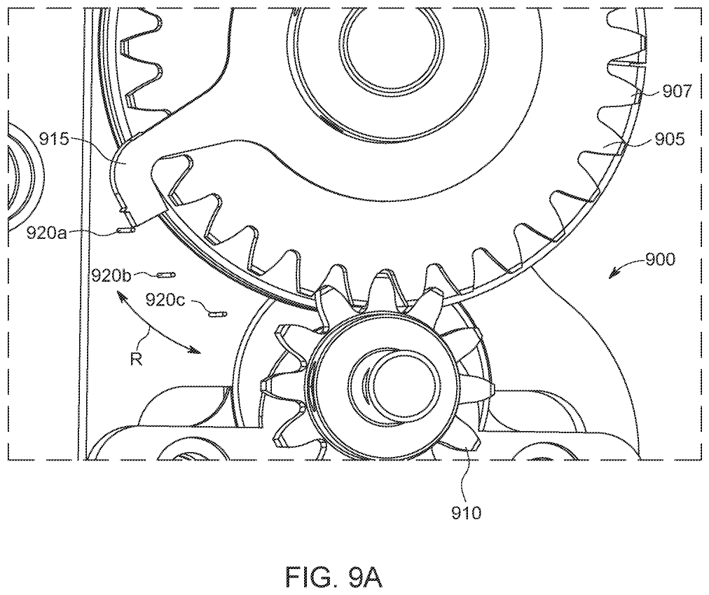

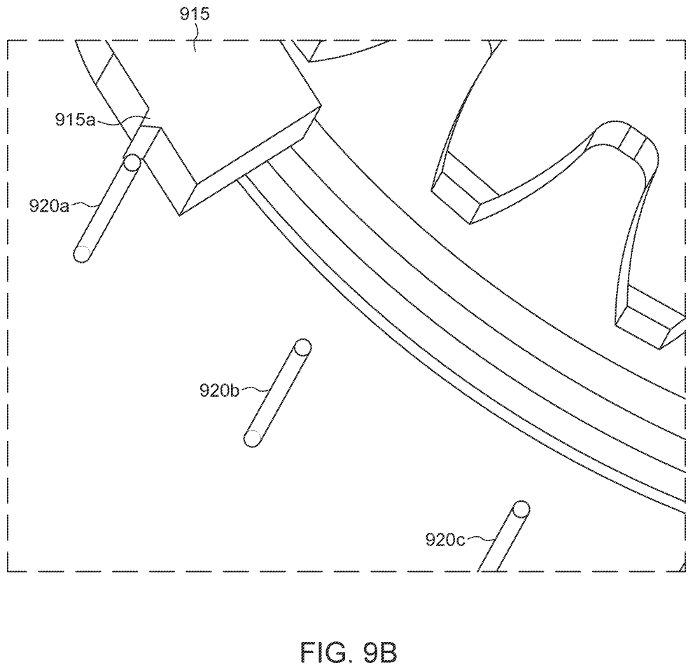

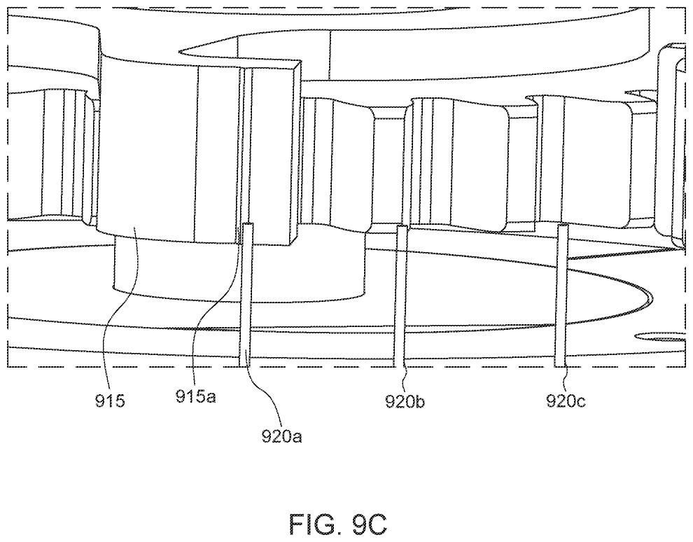

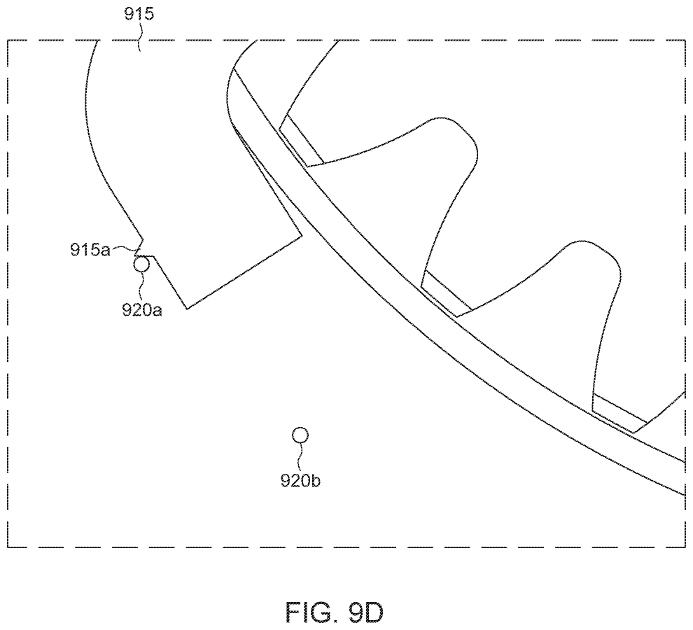

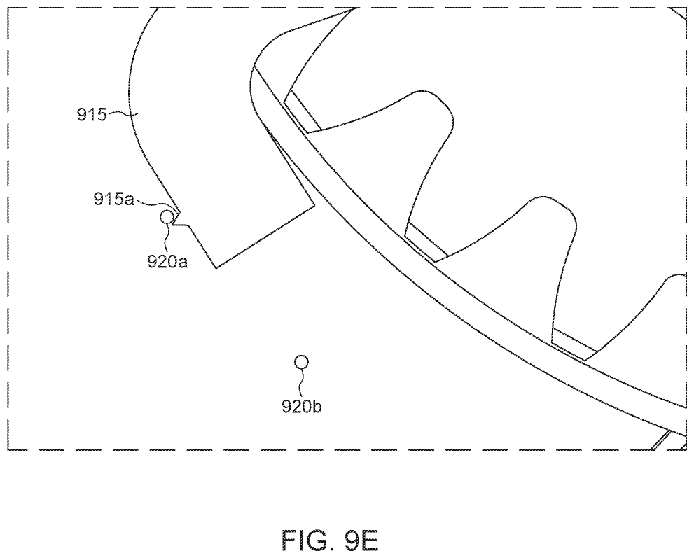

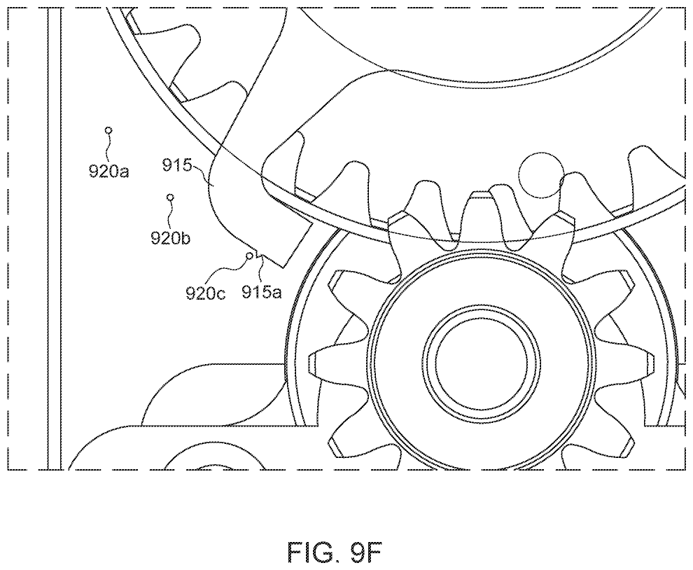

FIGS. 9A-9F illustrate a delivery feedback assembly 900 for use with a handle assembly (e.g., handle assembly 130). As described above, the steering catheter handle 132 includes one or more controls 134. The controls 134 can include a knob which, when rotated, cause rotation of one or more gears coupled to the knob. Rotation of such gears can apply tension to a pulley cable, which can result a change in curvature of the steering catheter. The pully cables may be also referred to as pull wires or steering cables, including those described above that pass through lumens 81 of steering catheter 80. For example, referring briefly again to FIG. 2A, rotating a control knob 134 in a first direction may tension a first steering cable passing through one pair of lumens 81 while simultaneously releasing tension on a second steering cable passing through a diametrically opposed pair of lumens 81. Rotating the control knob 134 in the opposite rotational direction can create tension on the second steering cable while releasing tension on the second steering cable. In some embodiments, one control knob 134 may be provided for each pair of diametrically opposed steering cables.

Referring again to FIGS. 9A-F. the delivery feedback assembly 900 can include a first gearset 905 and a second gearset 910. The first gearset 905 can rotate as shown by the arrows R and the first gearset 905 can include a hardstop mechanism 915 that rotates synchronously with the first gearset 905 as shown by the arrows R. The hardstop mechanism 915 can be any type of projection or other feature that extends radially outward relative to a perimeter defined by the gear teeth 907. In one example, the hardstop mechanism is L-shaped, but other shapes are contemplated depending upon the application.

Upon rotation of the first gearset 905 and the hardstop mechanism 915, the hardstop mechanism approaches the second gearset 910 until the hardstop mechanism 915 contacts the second gearset 910, resulting in interference between the hardstop mechanism 915 and the second gearset 910 and preventing further rotation of the first gearset 905. At this endpoint, continued rotation of the controls 134 (e.g., knob) is no longer possible. In operation, however, the endpoint is not always known to the operator prior to the hardstop mechanism 915 contacting the gearset 910.

Advantageously, the present delivery feedback system provides an operator with tactile and/or audible feedback regarding an angular position of the first gearset 905 and thus, a degree to which the steering catheter is curved.

Along the rotational path of the hardstop mechanism 915, the delivery feedback assembly 900 can include a plurality of feedback features 920 configured to selectively engage with a projection 915a on the hardstop mechanism 915. As the first gearset 905 rotates, a projection 915a of the hardstop mechanism 915 can engage first with a first feedback feature 920a. The engagement between the projection 915a and the first feedback feature 920a can provide tactile feedback to the operator, as the engagement will require additional force to advance the projection 915a and the hardstop mechanism 915 past the first feedback feature 920a.

Once the additional force is applied, the movement of the projection 915a past the first feedback feature 920a provides audible feedback by virtue of the disengagement between the two. In this regard, the first feedback feature 920a and/or the projection 915a can have some resilience such that the disengagement results in an audible click or other tone.

Upon further rotation, the projection 915a can engage with a second feedback feature 920b. The engagement between the projection 915a and the second feedback feature 920b can provide tactile feedback to the operator, as the engagement will require additional force to advance the projection 915a and the hardstop mechanism 915 past the second feedback feature 920b. Once the additional force is applied, the movement of the projection 915a past the second feedback feature 920b provides audible feedback by virtue of the disengagement between the two. In this regard, the second feedback feature 920b can have some resilience such that the disengagement results in an audible click or other tone. The audible and tactile feedback can provide the operator with information about angular displacement of the first gearset 905, where the angular displacement between first and second features 920a, b is known.

Upon further rotation, the projection 915a can engage first with a third feedback feature 920c. The engagement between the projection 915a and the third feedback feature 920c can provide tactile feedback to the operator, as the engagement will require additional force to advance the projection 915a and the hardstop mechanism 915 past the third feedback feature 920c. Once the additional force is applied, the movement of the projection 915a past the third feedback feature 920c provides audible feedback by virtue of the disengagement between the two. In this regard, the third feedback feature 920c can have some resilience such that the disengagement results in an audible click or tone. The audible and tactile feedback can provide the operator with information about angular displacement of the first gearset 905, where the angular displacement between first, second, and third features 920a-c is known. Upon passing the third and final feedback feature 920c, the operator knows he or she is approaching the endpoint. Thus, upon further rotation, the hardstop mechanism 915 will contact the second gearset 910, ceasing the angular displacement of the first gearset.

Similarly, the operator can counter rotate the knobs such that the first gearset rotates and passes from third feedback feature 920c to second feedback feature 920b to first feedback feature 920a. In some examples, each of the feedback features can provide differing audible or tactile feedback. For example, the pitch of the audible feedback may be different for each of the features. This may be achieved, for example, by forming each feedback feature 920a-c of the same material but having a different thickness. In another example, the pitch of the features may differ between rotation vs. counter rotation. In still another example, the force required to overcome features may differ.

While preparing a delivery system for use to deliver an IV device such as a prosthetic heart valve, it is typically desirable to purge all (or substantially all) air from the system prior to insertion of the delivery device into the patient. This is because, if air is introduced into the bloodstream during a procedure, the air may form a bubble capable of restricting blood flow through the vasculature, potentially resulting in an infarction. This is particularly dangerous for procedures being performed within a patient's left heart, since some of the first arterial branches from the left heart (e.g., the carotid arteries) lead to the brain. An air bubble flowing through the bloodstream in the path of the carotid arteries may result in a stroke. And it is not only important to purge air from the delivery device, but also to ensure that little or no air is later introduced into the delivery device during other preparatory procedures, such as loading a prosthetic heart valve into the catheter during a valve loading process.

In some systems, the only way in which an operator confirms that no air remains within a delivery system is via visual inspection. However, with some devices, particularly those made of metal or otherwise formed of opaque materials, a visual inspection is either impractical or impossible. Further, visual inspections are subjective and different operators having different experience may provide inconsistent confirmation of a complete purge. Some devices may provide for more objective measures of air bubbles residing within an otherwise flushed delivery device, such as ultrasonic air bubble sensors. While certain ultrasonic air bubble sensors exist, such sensors typically require clipping with respect to the object undergoing detection. Such clipping can cause permanent damage to metal catheter shafts, such as the various nested catheter layers (including the outer sheath 82) described in connection with delivery system 190. Further, there is no mechanism for detecting internal air bubbles along a length of a shaft, as the liquid inside the metal catheter shaft is not flowing, and ultrasonic air bubble detectors generally require fluid to be flowing through the sensor to be effective.

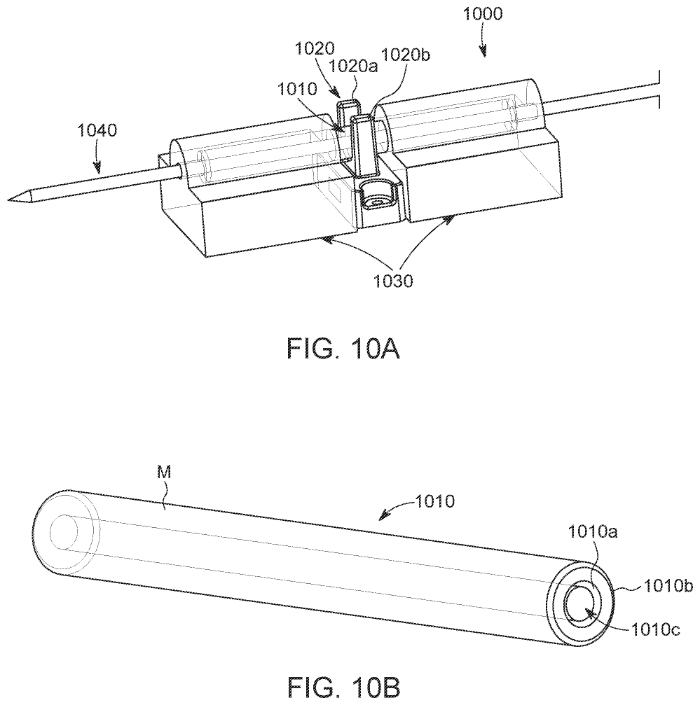

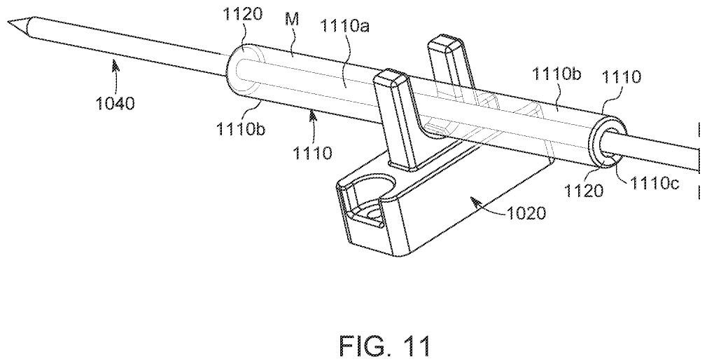

FIGS. 10A-B illustrate a non-contact air bubble detection assembly 1000 for scanning a length of a catheter shaft to detect air bubbles, including metal catheters shafts similar to those shown and described in connection with FIGS. 1-2A. The non-contact air bubble detection assembly 1000 advantageously provides a conformal surface so as not to damage the metal catheter and provides for a continuous contact medium to the metal catheter. This is advantageous because it allows the sensor or the catheter to move relative to each other without the catheter shaft incurring damage.

As shown in FIG. 10A, the non-contact air bubble detection assembly 1000 can include a double-walled fluid cylinder 1010 configured to receive a metal catheter shaft 1040 therethrough. The double-walled fluid cylinder 1010 can include an inner wall 1010a and an outer wall 1010b. An interior portion of the inner wall 1010a defines a lumen 1010c configured to receive the metal catheter shaft. Between the inner wall 1010a and the outer wall 1010b is a fluid-tight chamber that holds an ultrasonic-propagating medium M, such as water or saline. The lumen 1010c is open on both sides, allowing for a metal catheter shaft to pass completely therethrough. It should be understood that, while the air bubble detection assemblies described herein may be particularly useful with devices having metal catheter shafts, the assemblies may be used effectively with catheter shafts formed of other materials, including polymers.

The non-contact air bubble detection assembly 1000 can also include an ultrasonic bubble sensor 1020 configured to receive the double-walled fluid cylinder 1010 and a base 1030 to which the ultrasonic bubble sensor 1020 is mounted. In operation, the ultrasonic bubble sensor 1020 can detect the presence of air bubbles in the metal catheter shaft 1040 while the metal catheter shaft 1040 rests within the lumen 1010c by emitting an ultrasonic signal (e.g., from emitter portion 1020a) that passes through the medium M and the metal catheter shaft 1040. The emitted ultrasonic signal is detected (e.g., by detector portion 1020b) by the ultrasonic bubble sensor 1020 and, the presence of air bubbles, microbubbles, or other air in the metal catheter shaft can be detected by a change in impedance compared to the emitted signal. In this regard, the ultrasonic bubble sensor 1020 need not contact, and in one particular example does not contact, the metal catheter shaft 1040. Moreover, the metal catheter shaft 1040 need not contact, and in one particular example does not contact, the medium M held within the cylinder.

The metal catheter shaft 1040 can be passed through the lumen 1010c while the base 1030 (and also the ultrasonic bubble sensor 1020) is stationary, allowing the ultrasonic bubble sensor 1020 to detect bubbles along a portion or an entire length of the metal catheter shaft 1040. In another example, the metal catheter shaft 1040 is stationary while the base 1030 (and also the ultrasonic bubble sensor 1020) translates along a longitudinal direction of the metal catheter shaft 1040. In still another example, one or both of the metal catheter shaft 1040 and/or base 1030 (and also the ultrasonic bubble sensor 1020) can move relative to each other.

FIG. 11 illustrates a bubble detection assembly according to another aspect of the disclosure. In this example, the cylinder 1110 define an outer wall 1110b and a lumen 1110c. In this example, the cylinder 1110 does not include an inner wall, but can include hemostasis valves 1120 at each end that prevent a medium M from exiting the cylinder 1110. In this regard, the ultrasonic bubble sensor 1020 need not contact, and in one particular example does not contact, the metal catheter shaft 1040.

As in the prior example, the metal catheter shaft 1040 can be passed through the lumen 1110c while a base (not shown in FIG. 11) is stationary, allowing the ultrasonic bubble sensor 1020 to detect bubbles along a portion or an entire length of the metal catheter shaft 1040. In another example, the metal catheter shaft 1040 is stationary while the base (not shown in FIG. 11) translates along a longitudinal direction of the metal catheter shaft 1040. In still another example, one or both of the metal catheter shaft 1040 and/or base (not shown in FIG. 11) can move relative to each other. During any of these relative movements, the metal catheter shaft 1040 contacts the medium M contained within the cylinder 1110, with the hemostasis valves 1120 sealing around the catheter shaft 1040 during relative movement.

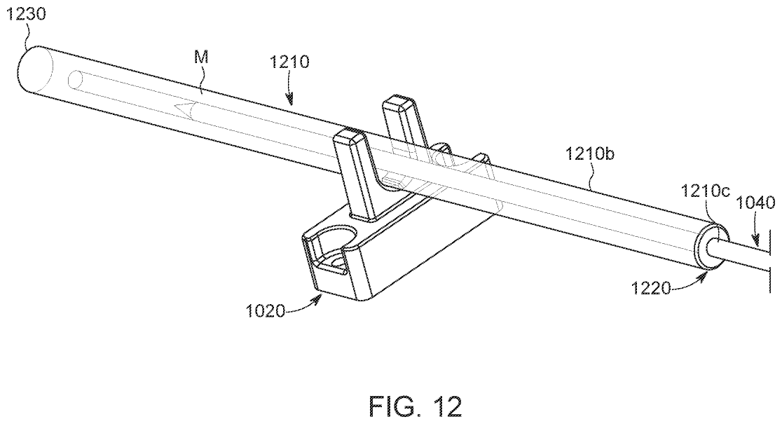

FIG. 12 illustrates a bubble detection assembly according to another aspect of the disclosure. In this example, the cylinder 1210 defines an outer wall 1210b and a lumen 1210c. In this example, the cylinder 1210 does not include an inner wall, but can include a hemostasis valve 1220 at one side and a closed end 1230 at the other side. In this regard, the lumen 1210c extends along a portion of the length of the cylinder 1210 and the hemostasis valve 1220 prevents a medium M from exiting the cylinder 1210. In this regard, the ultrasonic bubble sensor 1020 need not contact, and in one particular example does not contact, the metal catheter shaft 1040.

As in the prior examples, the metal catheter shaft 1040 can be passed through the lumen 1210c while a base (not shown in FIG. 12) is stationary, allowing the ultrasonic bubble sensor 1020 to detect bubbles along a portion or an entire length of the metal catheter shaft 1040. In another example, the metal catheter shaft 1040 is stationary while the base (not shown in FIG. 12) translates along a longitudinal direction of the metal catheter shaft 1040. In still another example, one or both of the metal catheter shaft 1040 and/or base (not shown in FIG. 12) can move relative to each other. During any of these relative movements, the metal catheter shaft 1040 contacts the medium M contained within the cylinder 1210.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A delivery system, comprising:
a handle;
a delivery member operably coupled to the handle and extending distally from the handle, the delivery member including an outer sheath, the outer sheath having a plurality of microfilaments braided together to form a braided coil; and
a distal tip at a distal end of the delivery member, the distal tip being configured to receive a medical device therein.

2. The delivery system of claim 1, wherein the plurality of microfilaments includes a first microfilament, a second microfilament, and a third microfilament.

3. The delivery system of claim 2, wherein the first microfilament has a first elasticity, the second microfilament has a second elasticity less than the first elasticity, and the third microfilament has a third elasticity less than both the first and second elasticities.

4. The delivery system of claim 2, wherein the plurality of microfilaments are made of the same material.

5. The delivery system of claim 4, wherein the material is nitinol.

6. The delivery system of claim 1, wherein the outer sheath includes a braided structure in addition to the braided coil, and a flexible cover that surrounds one or both of the braided structure and the braided coil.

7. The delivery system of claim 6, wherein the braided structure surrounds the braided coil.

8. The delivery system of claim 1, wherein the outer sheath includes a second coil, and a flexible cover that surrounds one or both of the braided coil and the second coil.

9. The delivery system of claim 1, wherein, when the outer sheath is in a bent condition, the plurality of microfilaments are configured to slide over each to reduce recoil as the outer sheath translates relative to an interior catheter of the delivery member during medical device deployment.

10. The delivery system of claim 1, wherein each of the plurality of microfilaments is circular in cross-section.

11. The delivery system of claim 1, wherein the delivery system includes the medical device, and the medical device is a prosthetic mitral valve.

12. A segmented nosecone, comprising:
a plurality of segments that, in an assembled condition, form the shape of a cone or a truncated cone, each of the plurality of segments including a positive alignment feature and a negative alignment feature configured to align the plurality of segments relative to each other;
a plurality of lips, each of the plurality of lips hingedly coupled with a respective one of the plurality of segments via a respective hinge.

13. The segmented nosecone of claim 12, wherein the plurality of segments, in the assembled condition, form a tip defining a hole.

14. The segmented nosecone of claim 13, wherein the plurality of segments, in the assembled condition, defines a lumen extending from a proximal end of the segmented nosecone to a distal end of the segmented nose cone.

15. The segmented nosecone of claim 14, wherein a first portion of the lumen defines a continuously decreasing diameter from a proximal end to a distal end of the first portion and a second portion of the lumen defines substantially constant diameter.

16. The segmented nosecone of claim 15, wherein the second portion of the lumen defines a substantially constant diameter approximately equal to an outer diameter of a guidewire.

17. The segmented nosecone of claim 14, wherein each of the plurality of segments includes a locking ring.

18. The segmented nosecone of claim 17, wherein the locking rings, in the assembled condition, align longitudinally to define a locking ring lumen configured to allow a guidewire to pass therethrough.

19. A delivery system, comprising:
a handle;
a delivery member operably coupled to the handle and extending distally from the handle;
a valve cover at a distal tip of the delivery member; and
the segmented nose cone of claim 12, the segmented nose cone being coupled to the valve cover by the plurality of lips.
